(19) United States
(12) Patent Application Publication
(10) Pub. No.: US 2019/0374118 A1
(43) Pub. Date: Dec. 12, 2019

(54) METHOD AND APPARATUS FOR PULSE WAVE MEASUREMENT

(71) Applicant: DENSO CORPORATION, Kariya-city (JP)

(72) Inventors: Tsuyoshi NAKAGAWA, Kariya-city (JP); Masanobu SUZUKI, Kariya-city (JP); Teruhiko KAMEOKA, Kariya-city (JP)

(21) Appl. No.: 16/433,442

(22) Filed: Jun. 6, 2019

(30) Foreign Application Priority Data
Jun. 8, 2018 (JP) 2018-110327

(51) Int. Cl. A61B 5/024; A61B 5/00

(52) U.S. Cl. CPC A61B 5/02416; A61B 5/7278; A61B 5/681; A61B 5/02438

(57) ABSTRACT

In a method for measuring a biometric signal of a subject, the biometric signal is repeatedly detected using a biometric sensor and a noise intensity of noise contaminating the detected biometric signal is detected. Further, in the method, a signal intensity of the detected biometric signal is adjusted if the signal intensity meets an intensity change condition. If the detected noise intensity is greater than a noise threshold, adjustment of the signal intensity is withheld.

### CROSS-REFERENCE TO RELATED APPLICATION

This application is based on and claims the benefit of priority from earlier Japanese Patent Application No. 2018-110327 filed on Jun. 8, 2018, the description of which is incorporated herein by reference.

### BACKGROUND

### Technical Field

This disclosure relates to a signal measurement technique using a sensor.

### Related Art

An intensity of a signal measured by a sensor may vary due to conditions of an object to be measured or other factors. Variations in signal intensity may cause errors in processing. A known pulse wave measurement apparatus is configured to set a gain change condition and change a gain of the pulse wave sensor when an amplitude of the pulse wave signal acquired by the pulse wave sensor meets the gain change condition, thereby bringing the intensity of the pulse wave signal into a certain range.

However, there is a need for a signal measurement method that can inhibit unnecessary adjustment of the signal intensity.

### SUMMARY

One aspect of the disclosure provides a method for measuring a biometric signal of a subject. In the method, the biometric signal is repeatedly detected using a biometric sensor and a noise intensity of noise contaminating the detected biometric signal is detected. Further, in the method, a signal intensity of the detected biometric signal is adjusted if the signal intensity meets an intensity change condition. If the detected noise intensity is greater than a noise threshold, adjustment of the signal intensity is withheld.

### BRIEF DESCRIPTION OF THE DRAWINGS

In the accompanying drawings:

FIG. 1 is a block diagram of a pulse wave measurement apparatus according to a first embodiment;

FIG. 2 is a flowchart of signal measurement processing according to the first embodiment;

FIG. 3 is an example of a time variation of a pulse wave signal;

FIG. 4 is a frequency spectrum of a measured signal where the intensity of an untargeted signal is greater than the intensity of a targeted signal;

FIG. 5 is a frequency spectrum of a measured signal where the intensity of a targeted signal is greater than the intensity of an untargeted signal;

FIG. 6 is an example of a time variation of a pulse wave signal with no intensity adjustment performed;

FIG. 7 is an example of a time variation of a pulse wave signal with intensity adjustment performed;

FIG. 8 is a flowchart of signal measurement processing according to a second embodiment;

FIG. 9 is an example of accelerations along three axes and the magnitude of body motion calculated from the accelerations along the three axes;

FIG. 10 is an enlarged view of a range R shown in FIG. 9;

FIG. 11 is an example of a time variation of the pulse wave signal affected by the body motion;

FIG. 12 is an example of a time variation of the magnitude of body motion; and

FIG. 13 is a flowchart of signal measurement processing according to a third embodiment.

### DESCRIPTION OF SPECIFIC EMBODIMENTS

Hereinafter, exemplary embodiments of the present disclosure will be described in detail with reference to the accompanying drawings, in which like reference numerals refer to like or similar elements regardless of reference numerals and duplicated description thereof will be omitted.

### First Embodiment

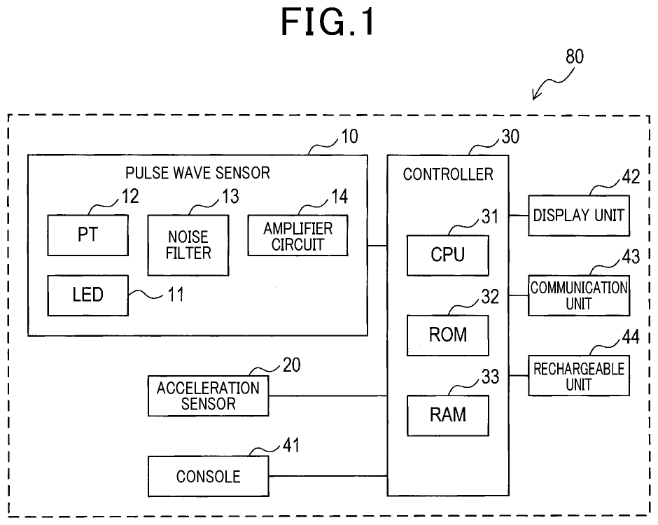

A pulse wave measurement apparatus 80 according to a first embodiment will now be described with reference to FIG. 1. The pulse wave measurement apparatus 80 includes a pulse wave sensor 10, an acceleration sensor 20, a controller 30, a console 41, a display unit 42, a communication unit 43, and a rechargeable unit 44. In the present embodiment, the pulse wave measurement apparatus 80 may be a wrist-wearable device, such as a watch.

The pulse wave sensor 10 is configured to detect pulse wave signals from part of a subject, such as a finger, a wrist or the like. A pulse wave signal is a signal representing a pulse wave, more specifically, a signal measured reflecting changes in blood flow in the vicinity of part of the subject where the pulse wave is measured. In the present embodiment, the pulse wave sensor 10, which is incorporated in the pulse wave measurement apparatus 80 and attached to a subject's wrist, is configured to detect a pulse wave signal from the wrist of the subject.

The pulse wave sensor 10 includes a light-emitting diode (LED) 11, a phototransistor (PT) 12, a noise filter 13, and an amplifier circuit 14. In the present embodiment, the LED 11 serves as a light emitting element. The PT 12 serves as a light receiving element.

The LED 11 is configured to irradiate a skin of the subject with an amount of emitted visible light, that is, an output intensity of light, indicated by the controller 30. The wavelength of the emitted visible light is within a range of 5000 Å to 7000 Å. Part of irradiation light emitted from the LED 11 is reflected from a skin capillary, which causes reflected light. The PT 12 receives the reflected light generated in the skin capillary to generate and output an electrical signal to the noise filter 13. The generated electrical signal is a pulse wave signal that varies reflecting a pulse wave of the subject. In place of the PT 12, a photodiode may be used.

The noise filter 13 removes noise, such as white noise, included in the pulse wave signal, and outputs the pulse wave signal having noise removed to the amplifier circuit 14. The amplifier circuit 14 amplifies the pulse wave signal with an amplification factor A (hereinafter, a gain A) indicated by the controller 30 and transmits the amplified pulse wave signal to the controller 30. The controller 30 stores the received pulse wave signal in the memory.

The acceleration sensor 20 is configured to detect an acceleration signal representing a direction and a magnitude of acceleration in each of the X-axis direction, the Y-axis direction, and the Z-axis direction, and transmits the detected acceleration signal to the controller 30. The controller 30 stores the received acceleration signal in the memory. In the present embodiment, the acceleration sensor 20 is incorporated in the pulse wave measurement apparatus 80 and attached to the wrist of the subject, and detects a direction and a magnitude of acceleration of a subject's body.

The console 41 undergoes user's manipulations and outputs signals corresponding to the manipulations to the controller 30. The user's manipulations may include a manipulation to direct the user to initiate pulse wave measurement. The user may be a subject himself/herself or medical personnel that assists the subject.

The display unit 42 is able to display images. The communication unit 43 is provided for wireless or wired communications with the exterior of the pulse wave measurement apparatus 80. The power source 44 supplies power to each part of the pulse wave measurement apparatus 80.

The controller 30 may be configured as one or more microcomputers including a central processing unit (CPU) 31, a read-only memory (ROM) 32, a random-access memory (RAM) 33, a memory, an input-output interface, and other components. Various functions of the controller 30 may be implemented by the CPU 31 loading and executing computer programs stored in the ROM 32 that serves as a non-transitory computer readable storage medium in the present embodiment. A pulse waveform analyzing method described later may be implemented by the CPU 31 executing the programs. Functions of the controller 30 may be implemented by software only, hardware only, or a combination thereof. For example, when these functions are provided by an electronic circuit which is hardware, the electronic circuit can be provided by a digital circuit including many logic circuits, an analog circuit, or a combination thereof.

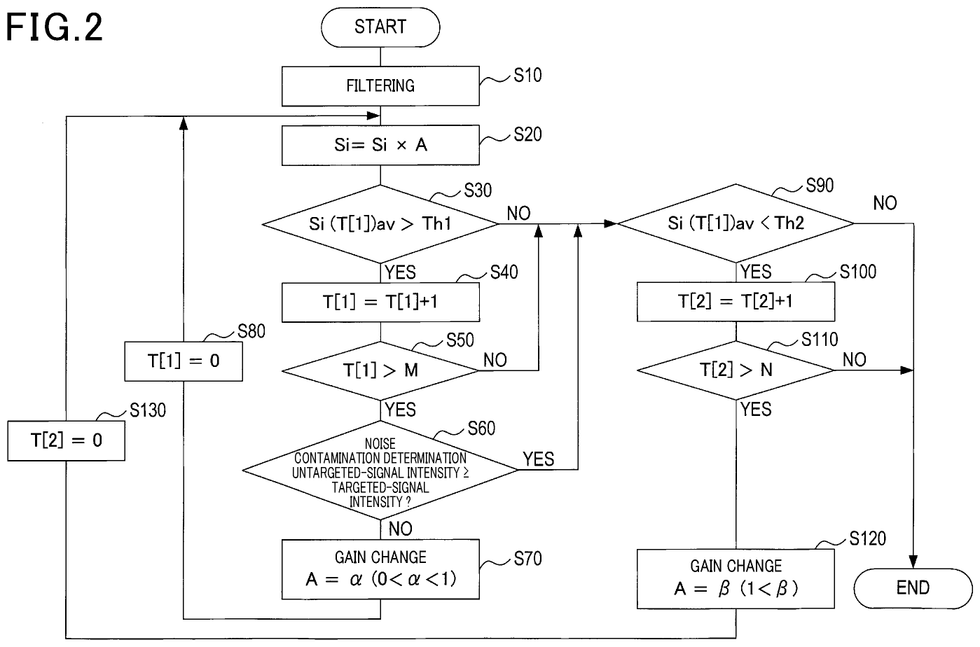

Measurement processing of pulse wave signals performed by the pulse wave measurement apparatus 80 will now be described with reference to a flowchart of FIG. 2. The pulse wave measurement apparatus 80 performs this processing every predetermined time interval ΔT.

At step S10, the pulse wave sensor 10 applies the noise filter 13 to a pulse wave signal Si extracted from the received reflected light, thereby filtering the pulse wave signal.

Subsequently, at step S20, the pulse wave sensor 10 amplifies the filtered pulse wave signal Si with the gain A via the amplifier circuit 14. The gain A is set to one if the pulse wave sensor 10 does not perform intensity adjustment of the pulse wave signal Si. The gain A is set to a (0<α<1) or β (1<β) as described later if the pulse wave sensor 10 performs intensity adjustment of the pulse wave signal Si.

Unless a specific condition as described later is met, the intensity adjustment of the pulse wave signal Si, in which the signal intensity Ia of the pulse wave signal Si is adjusted, is performed if the signal intensity Ia meets one of the intensity change conditions. The intensity change conditions include the following conditions (i) and (ii). The intensity change condition (i) is that an average value of peaks of the signal intensity Ia as described later continues to be above a first intensity threshold Th1 for a first period of determination M. The intensity change condition (ii) is that an average value of peaks of the signal intensity Ia continues to be below a second intensity threshold Th2 for a second period of determination N. The signal intensity Ia of the pulse wave signal Si above the first intensity threshold Th1 leads to saturation of the pulse wave signal Si where the pulse wave signal Si can not be detected. The signal intensity Ia below the second intensity threshold Th2 leads to a low resolution of the signal intensity Ia.

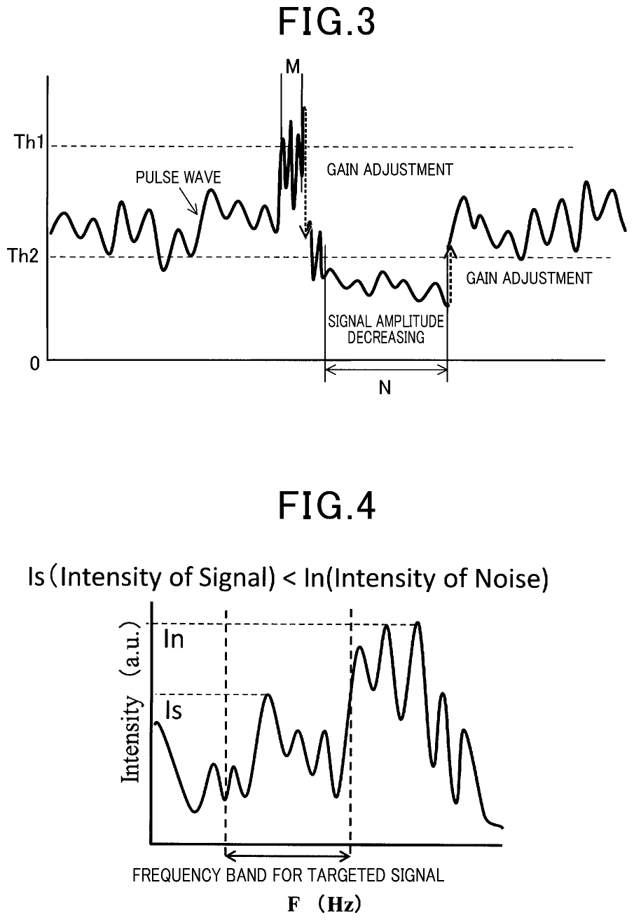

Therefore, as shown in FIG. 3, if the intensity change condition (i) is met, the gain A is set to less than one and the signal intensity Ia is thereby decreased. For example, when a subject moves from a cold place to a warm place, the blood flow of the subject may increase and thus the signal intensity Ia may increase, which may cause the signal intensity Ia to meet the intensity change condition (i). In addition, as shown in FIG. 3, if the intensity change condition (ii) is met, the gain A is set to greater than one and the signal intensity Ia is thereby increased.

Subsequently, at step S30, the controller 30 determines whether or not an average Si(T[1])av is greater than a first intensity threshold Th1. T[1] represents time and an initial value of T[1] is zero. The average Si(T[1]) at time T[1] is an average value of peaks of the signal intensity Ia taken over an unit time ΔT that is an interval between the current and previous cycles. If the average Si(T[1])av is equal to or less than the first intensity threshold Th1, the controller 30 proceeds to step S90 because the intensity change condition (i) can not be met.

If the average Si(T[1])av is greater than the first intensity threshold Th1, the intensity change condition (i) may be met. The controller 30 then proceeds to step S40 and updates the time T[1] to T[1]+1. That is, the controller 30 acquires a duration in which the average Si(T[1]) is greater than the first intensity threshold Th1.

Subsequently, at step S50, the controller 30 determines whether or not the time T[1] is greater than the first period of determination M. That is, the controller 30 determines whether or not the intensity change condition (i) is met. If the time T[1] is equal to or less than the first period of determination M, then the intensity change condition (i) is not met and thus the controller 30 proceeds to step S90.

If the time T[1] is greater than the first period of determination M, then the intensity change condition (i) is met and thus the controller 30 proceeds to step S60. At step S60, the controller 30 determines whether or not a specific condition (a) is met.

More specifically, the specific condition (a) is that, for the pulse wave signal Si, a maximum peak value of the signal intensity In of untargeted noise is greater than a maximum peak value of the signal intensity Is of the targeted pulse wave signal. Noise is a signal outside of a frequency band F intended for targeted pulse wave signals. A targeted pulse wave signal is a signal whose frequency is within the frequency band F.

While filtering of the pulse wave signal Si is performed at step S10, the amplitude of the pulse wave signal Si may be large due to noise superimposed on the pulse wave signal. In such a case, when the gain A is decreased in response to the intensity change condition (i) being met, the signal intensity Ia of the pulse wave signal Si having noise removed may be small. Consequently, when noise superimposed on the pulse wave signal disappears, the signal intensity Ia of the pulse wave signal Si will fall below the second intensity threshold Th2 and thus the gain A will need to be changed again. That is, when the pulse wave signal Si is contaminated with noise such that the signal intensity Ia is greater than the first intensity threshold Th1, the controller 30 will have to make an otherwise unnecessary change of the gain A twice.

In light of the foregoing, the controller 30 is configured to, if the signal intensity Ia is greater than the first intensity threshold Th1 due to untargeted noise superimposed on the pulse wave signal, withhold changing the gain A even if the intensity change condition (i) is met. In response to whether the specific condition (a) is met, the controller 30 determines whether or not the pulse wave signal Si is contaminated with noise such that the signal intensity Ia is greater than the first intensity threshold Th1. That is, the controller 30 withholds adjustment of the signal intensity if the specific condition (a) is met.

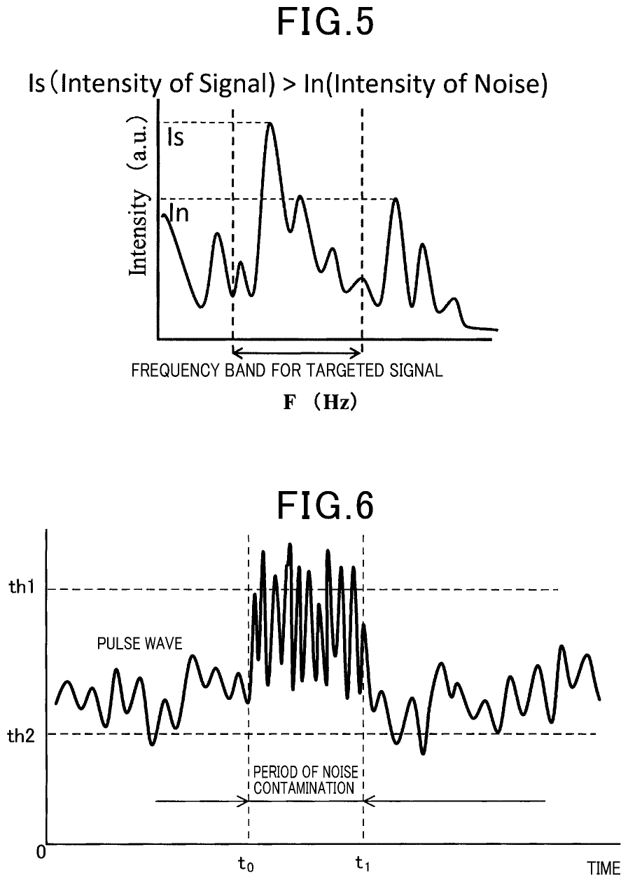

The controller 30 performs frequency analysis, such as fast Fourier transformation (FFT) analysis, of the pulse wave signal Si. Based on a result of the frequency analysis, the controller 30 detects a maximum peak value of the signal intensity Is within the frequency band F and a maximum peak value of the signal intensity In outside of the frequency band F, and sets the maximum peak value of the signal intensity Is as a signal threshold. If, as shown in FIG. 4, the maximum peak value of the signal intensity In is greater than the signal threshold, the controller 30 determines that the pulse wave signal Si is contaminated with noise. If, as shown in FIG. 5, the maximum peak value of the signal intensity In is less than the signal threshold, the controller 30 determines that the pulse wave signal Si is not contaminated with noise.

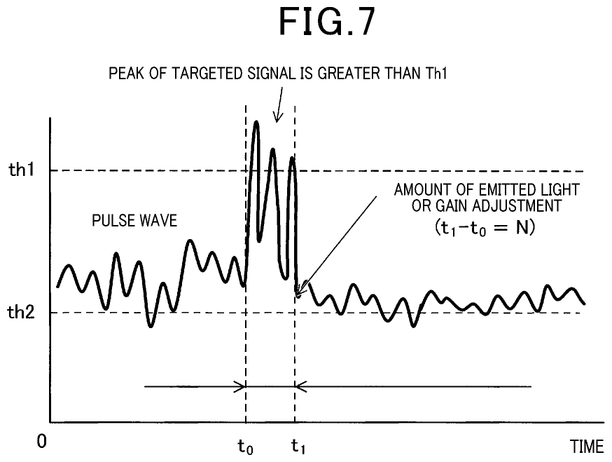

If at step S60 the controller 30 determines that the pulse wave signal Si is contaminated with noise, the controller 30 proceeds to step S90. FIG. 6 illustrates an example of time variations of the pulse wave signal Si in the case where it is determined that the pulse wave signal Si is contaminated with noise. If at step S60 the controller 30 determines that the pulse wave signal Si is not contaminated with noise, the controller 30 proceeds to step S70. FIG. 7 illustrates an example of time variations of the pulse wave signal Si in the case where it is determined that the pulse wave signal Si is not contaminated with noise.

At step S70, the controller 30 changes the gain A to α. α tales a value greater than zero and less than one. Subsequently, at step S80, the controller 30 resets the time T[1] to zero and returns to step S20.

Subsequently, at step S90, the controller 30 determines whether or not an average Si(T[2])av is less than a second intensity threshold Th2. T[2] represents time and an initial value of T[2] is zero. The average Si(T[2]) at time T[2] is an average value of peaks of the signal intensity Ia taken over an unit time ΔT that is an interval between the current and previous cycles. If the average Si(T[2])av is equal to or greater than the second intensity threshold Th2, the controller 30 ends this processing without changing the gain A because the intensity change condition (ii) can not be met.

If the average Si(T[2])av is less than the second intensity threshold Th2, the intensity change condition (ii) may be met. The controller 30 then proceeds to step S100 and updates the time T[2] to T[2]+1. That is, the controller 30 acquires a duration in which the average Si(T[2]) is less than the second intensity threshold Th2.

Subsequently, at step S110, the controller 30 determines whether or not the time T[2] is greater than the second period of determination N. That is, the controller 30 determines whether or not the intensity change condition (ii) is met. If the time T[2] is equal to or less than the second period of determination N, then the intensity change condition (ii) is not met and thus the controller 30 ends the processing without changing the gain A.

If the time T[2] is greater than the second period of determination N, then the intensity change condition (ii) is met and thus the controller 30 proceeds to step S120.

At step S120, the controller 30 changes the gain A to R. 13 takes a value greater than one. Subsequently, at step S130, the controller 30 resets the time T[2] to zero and returns to step S20.

In the present embodiment, the controller 30 is configured to perform intensity adjustment of the pulse wave signal Si by changing the gain A. In one alternative embodiment, the controller 30 may be configured to perform intensity adjustment of the pulse wave signal Si not by changing the gain A, but by changing an amount of light emitted from the LED 11. That is, instead of multiplying the gain A by a factor α or β, the controller 30 may multiply an amount of light emitted from the LED 11 by a factor α or β. The signal intensity Ia of the pulse wave signal Si can be decreased by decreasing the amount of light emitted from the LED 11. The signal intensity Ia of the pulse wave signal Si can be increased by increasing the amount of light emitted from the LED 11. In another alternative embodiment, the controller 30 may be configured to change both the gain A and the amount of light emitted from the LED 11 to adjust the signal intensity Ia of the pulse wave signal Si to bring the signal intensity Ia of the pulse wave signal Si into a range of the second intensity threshold Th2 to the first intensity threshold Th1.

Advantages

The present embodiment configured as above can provide the following advantages.

(1) If the signal intensity In outside of the frequency band F is greater than the signal intensity Is within the frequency band F, the signal intensity Ia of the pulse wave signal Si is likely to be greater than the first intensity threshold Th1 due to untargeted noise superimposed on the pulse wave signal. In such a case, adjustment of the signal intensity Ia of the pulse wave signal Si is withheld, which can inhibit unnecessary adjustment of the signal intensity.

(2) The signal intensity of the pulse wave signal Si varies according to the gain A of the amplifier circuit 14 and the amount of light emitted from the LED 11. Therefore, the signal intensity of the pulse wave signal Si can be adjusted by adjusting at least one of the gain A of the amplifier circuit 14 and the amount of light emitted from the LED 11.

### Second Embodiment

A second embodiment is similar in configuration to the first embodiment. Therefore, only differences of the second embodiment from the first embodiment will be described. Elements having the same functions as those in the first embodiment are assigned the same reference numerals and thus will not be redundantly described.

In the above first embodiment, noise contaminating the pulse wave signal Si is untargeted noise outside of the frequency band F. The second embodiment is different from the first embodiment in that noise contaminating the pulse wave signal Si is noise caused by body motion of the subject.

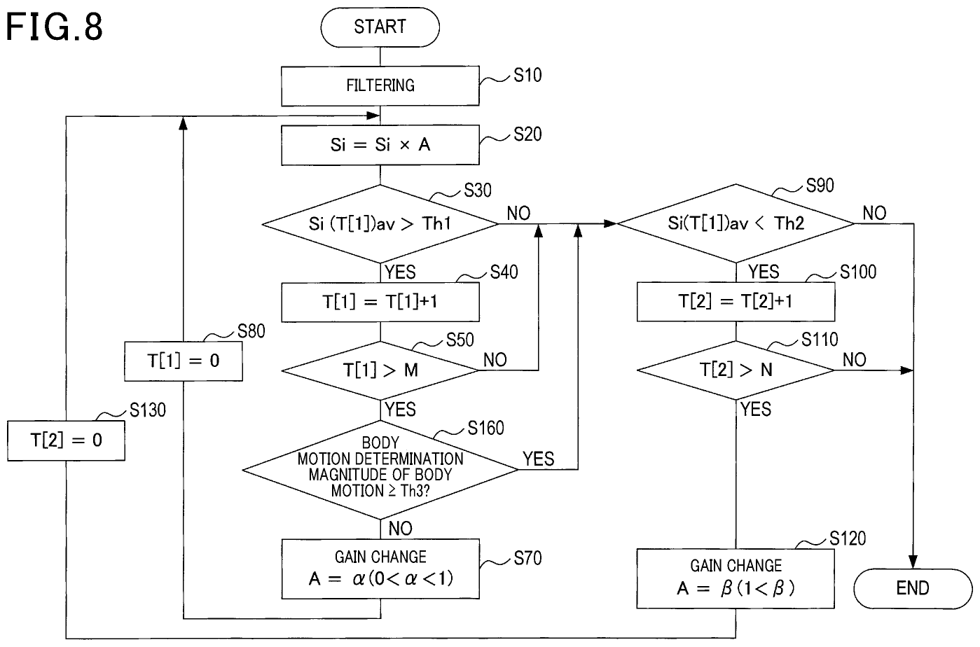

Measurement processing performed by the pulse wave measurement apparatus 80 of the second embodiment will now be described with reference to a flowchart of FIG. 8. According to the flowchart of FIG. 8, the pulse wave measurement apparatus 80 executes steps S10 to S50 and steps S70 to S130 in the flowchart of FIG. 2. The pulse wave measurement apparatus 80 executes step S160 instead of executing step S60 in the flowchart of FIG. 2.

At step S160, the controller 30 determines whether or not a specific condition (b) is met. More specifically, the specific condition (b) is that the magnitude of body motion at the current time is equal to or greater than a body motion threshold Th3. The body motion threshold Th3 is a predetermined value. For example, the magnitude of body motion and the pulse wave signal may be experimentally acquired. The body motion threshold Th3 may be set to a minimum of the magnitude of body motion that can affect the pulse wave signal.

Occurrence of a relatively large body motion may lead to an increased amplitude of the pulse wave signal Si due to the effects of body motion. In such a case, when the gain A is decreased in response to the intensity change condition (i) being met, the signal intensity Ia of the pulse wave signal Si may fall below the second intensity threshold Th2 when the effects of body motion has vanished. Thus the gain A may need to be changed again. That is, in a case where the signal intensity Ia of the pulse wave signal Si is greater than the first intensity threshold Th1 due to the effects of body motion, the controller 30 may have to make an otherwise unnecessary change of the gain A twice.

In light of the foregoing, the controller 30 is configured to, if the signal intensity Ia is greater than the first intensity threshold Th1 due to the effects of body motion, withhold changing the gain A even if the intensity change condition (i) is met. Depending on whether or not the specific condition (b) is met, the controller 30 determines whether or not the signal intensity Ia is greater than the first intensity threshold Th1 due to the effects of body motion. That is, the controller 30 withholds adjustment of the signal intensity if the specific condition (b) is met.

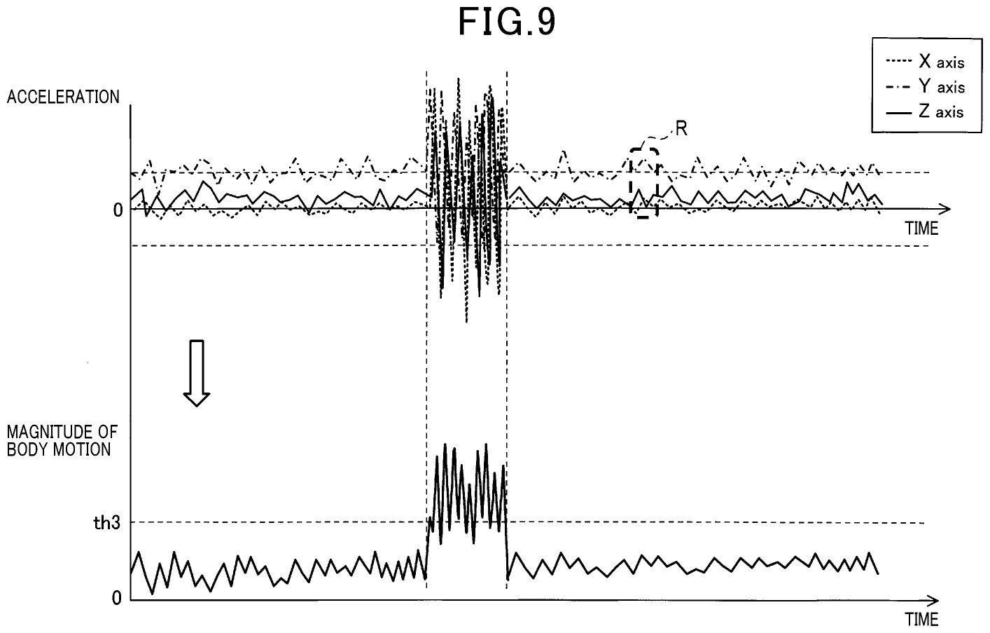

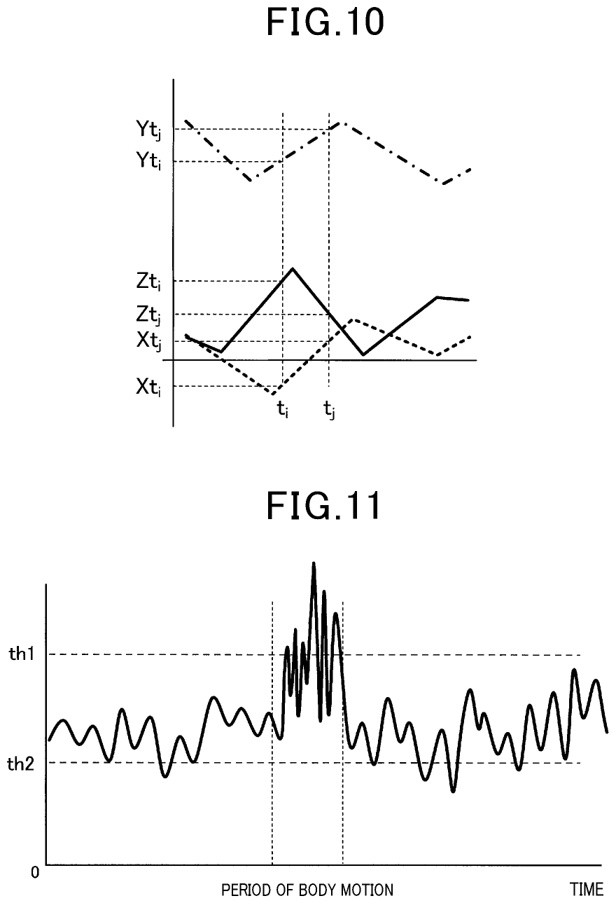

The controller 30 acquires an acceleration along each of three axes (X-axis, Y-axis and Z-axis) at the current time from the acceleration sensor 20. The upper part of FIG. 9 and FIG. 10 illustrate time variations of the acceleration along each of three axes. FIG. 10 is an enlarged view of a range R shown in the upper part of FIG. 9. The acceleration along each of three axes takes a value responsive to gravity in a case where there is no body motion. Occurrence of a body motion may lead to large time variations in the acceleration along each of three axes. The controller 30 calculates the magnitude of the body motion using the acceleration along each of the three axes and the following equation (1). In the equation (1), time tj is later than time ti. The lower part of FIG. 9 illustrates time variations of the magnitude of body motion calculated from the acceleration along each of the three axes shown in the upper part of the FIG. 9.

Magnitude of Body Motion=\sqrt{(Xtᵢ−Xtⱼ)²+(Ytᵢ−Ytⱼ)²+(Ztᵢ−Ztᵢ)²} (1)

If the magnitude of body motion is equal to or greater than the body motion threshold Th3, the controller 30 determines that there are effects of body motion and then proceeds to step S90. If the magnitude of body motion is less than the body motion threshold Th3, the controller 30 determines that there are no effects of body motion and then proceeds to step S70. In one alternative embodiment, as in the first embodiment, the controller may be configured to change the amount of light emitted from the LED 11 instead of changing the gain A or to change both the gain A and the amount of light emitted from the LED 11.

Advantages

The second embodiment can provide the following advantage in addition to the advantages of the first embodiment.

(3) If the magnitude of body motion is greater than the body motion threshold Th3, the signal intensity Ia of the pulse wave signal Si is likely to be greater than the first intensity threshold Th1 due to the effects of body motion. In such a case, adjustment of the signal intensity Ia of the pulse wave signal Si is withheld, which can inhibit unnecessary adjustment of the signal intensity.

### Third Embodiment

A third embodiment is similar in configuration to the second embodiment. Therefore, only differences of the third embodiment from the second embodiment will be described. Elements having the same functions as those in the second embodiment are assigned the same reference numerals and thus will not be redundantly described.

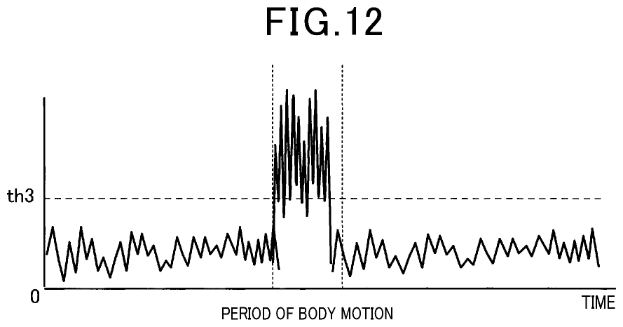

The third embodiment is different from the second embodiment in that a delay time from occurrence of a body motion until the body motion affects the pulse wave signal Si is taken into consideration. For example, the pulse wave affected by a motion of a wrist of the subject can immediately propagate to the pulse wave measurement apparatus 80 worn on the wrist of the subject. However, the pulse wave affected by a motion of a shoulder of the subject takes some time to propagate to the pulse wave measurement apparatus 80. FIG. 12 illustrates a time variation of the magnitude of the body motion. FIG. 11 illustrates a time variation of the pulse wave signal Si that is affected by the body motion after a predetermined time has elapsed from occurrence of the body motion as shown in FIG. 12.

That is, when a body motion of the subject has occurred, the amplitude of the pulse wave of the subject is likely to increase due to the effects of body motion within a predetermined delay time t after occurrence of the body motion. Thus, in the third embodiment, a delay time from occurrence of a body motion until the body motion affects the pulse wave signal Si is taken into consideration.

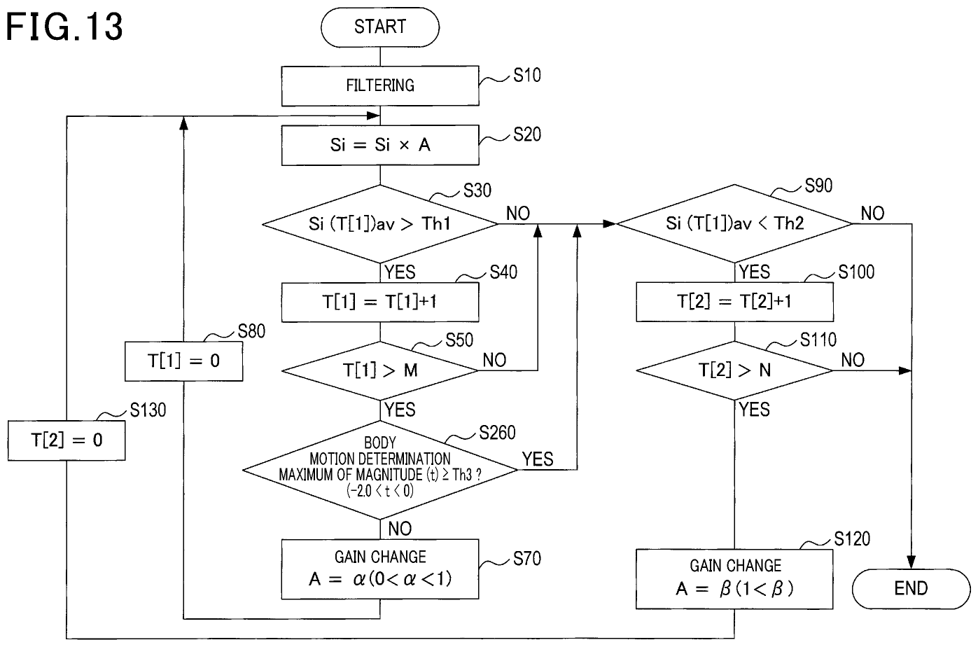

Measurement processing performed by the pulse wave measurement apparatus 80 of the third embodiment will now be described with reference to a flowchart of FIG. 13. According to the flowchart of FIG. 13, the pulse wave measurement apparatus 80 executes steps S10 to S50 and steps S70 to S130 in the flowchart of FIG. 2. The pulse wave measurement apparatus 80 executes step S260 instead of executing step S160 in the flowchart of FIG. 2.

At step S260, based on whether or not the specific condition (c) is met, the controller 30 determines whether or not the signal intensity Ia is greater than the first intensity threshold Th1 due to the effects of body motion. That is, the controller 30 withholds adjustment of the signal intensity if the specific condition (c) is met.

More specifically, the specific condition (c) is that a maximum of the magnitude of the body motion is equal to or greater than the body motion threshold Th3 within a detection time from a predetermined delay time t before the current time to the current time. For example, the magnitude of body motion and the pulse wave signal may be experimentally acquired. The delay time t may be set to a maximum period of time from occurrence of the body motion until the body motion affects the pulse wave signal. The delay time t may be set to one second or more, for example, two seconds. The controller 30 calculates the magnitude of the body motion within the detection time to acquire the maximum of the magnitude of the body motion.

If the maximum of the magnitude of the body motion is equal to or greater than the body motion threshold Th3, the controller 30 determines that the body motion affects the pulse wave signal and then proceeds to step S90. If the maximum of the magnitude of the body motion is less than the body motion threshold Th3, the controller 30 determines that the body motion does not affect the pulse wave signal and then proceeds to step S70. In one alternative embodiment, as in the first embodiment, the controller may be configured to change the amount of light emitted from the LED 11 instead of changing the gain A or to change both the gain A and the amount of light emitted from the LED 11 at step S70.

Instead of the predetermined value, a learned value may be used for each of the delay time t and the body motion threshold Th3. That is, the controller 30 may be configured to sequentially detect the magnitude of the body motion and learn the delay time using the detected magnitude of the body motion and the pulse wave signal, and set the delay time t to a learned value of the delay time. In addition, the controller 30 may be configured to sequentially detect the magnitude of the body motion and learn the body motion threshold using the detected magnitude of the body motion and the pulse wave signal, and set the body motion threshold Th3 to a learned value of the body motion threshold.

Advantages

The third embodiment can provide the following advantage in addition to the advantages (2) and (3) of the first embodiment.

(4) Comparing the maximum of the magnitude of the body motion within the detection time and the body motion threshold Th3 enables properly determining whether or not the body motion that affects the pulse wave signal Si has occurred, thereby inhibiting the presence or absence of the effects of body motion from being incorrectly determined.

(5) There may be a delay time from occurrence of a body motion until the pulse wave affected by the body motion propagates to measuring part. Therefore, setting the detection time from the delay time t before the current time to the current time can inhibit the presence or absence of the effects of body motion on the pulse wave signal Si from being incorrectly determined.

(6) A variation in blood flow caused by the body motion varies between individuals. Learning the delay time t enables setting the delay time t suited for each subject and can thus properly inhibit unnecessary adjustment of the signal intensity for characteristics of each individual.

(7) A variation in blood flow caused by the body motion varies between individuals. Learning the body motion threshold Th3 enables setting the body motion threshold Th3 suited for each subject and can thus properly inhibit unnecessary adjustment of the signal intensity for characteristics of each individual.

In summary, according to the above first to third embodiments, when the intensity of noise contaminating a biometric signal is greater than a noise threshold, adjustment of the signal intensity is withheld. That is, when the intensity of the biometric signal is increased due to effects of noise, adjustment of the signal intensity is withheld, which can inhibit the intensity of the biometric signal having noise components removed from decreasing. Therefore, when the effects of noise vanish, there is no need to readjust the signal intensity to increase the intensity of the biometric signal, which can inhibit unnecessary adjustment of the signal intensity.

Modifications

Specific embodiments of the present disclosure have so far been described. However, the present disclosure should not be construed as being limited to the foregoing embodiments, but may be modified in various modes.

(M1) At least two of the first embodiment, the second embodiment, and the third embodiment may be combined. That is, both a signal outside of the frequency band F and a body motion may be taken into consideration as noise contaminating the pulse wave signal Si. In such an embodiment, the controller 30 may be configured to execute step S160 or S260 after executing step S60 in the flowchart of FIG. 2.

(M2) In each of the above embodiments, pulse wave information is acquired by the photoelectric pulse wave sensor 10. In one alternative embodiment, an impedance-based sensor that detects changes in voltage caused by changes in biological impedance responsive to propagation of the pulse wave may be used to acquire pulse wave information. In another embodiment, pulse wave information may be acquired by detecting changes in luminance value responsive to propagation of the pulse wave from a captured image of a blood vessel of the subject.

(M3) In each of the above embodiments, the pulse wave sensor 10 and the acceleration sensor 20 are incorporated and integrated in the pulse wave measurement apparatus 80. In one alternative embodiment, the pulse wave sensor 10 and the acceleration sensor 20 may be separate devices. For example, the pulse wave sensor 10 may be incorporated in the pulse wave measurement apparatus 80 and the acceleration sensor 20 may be an external device to the pulse wave measurement apparatus 80. The acceleration sensor 20 may include a communication unit that can communicate with the communication unit 43 incorporated in the pulse wave measurement apparatus 80. Separating the pulse wave sensor 10 and the acceleration sensor 20 allows the pulse wave sensor 10 and the acceleration sensor 20 to be placed at different points on the subject's body. This configuration is effective when a point of measurement of the pulse wave and a point of occurrence of the body motion are different.

(M4) In each of the above embodiments, the pulse wave measurement method has been described. The pulse wave measurement method according to the present disclosure is applicable to a method for measuring biometric signals detected by biometric sensors other than the pulse wave signals. The biometric sensors may include a sensor that detects electrocardiogram and a sensor that detects brain waves. The biometric signals may include electrocardiogram signals and brain wave signals.

(M5) The functions of a single component may be distributed to a plurality of components, or the functions of a plurality of components may be integrated into a single component. At least part of the configuration of the above embodiments may be replaced with a known configuration having a similar function. At least part of the configuration of the above embodiments may be removed. At least part of the configuration of one of the above embodiments may be replaced with or added to the configuration of another one of the above embodiments. While only certain features of the invention have been illustrated and described herein, many modifications and changes will occur to those skilled in the art. It is, therefore, to be understood that the appended claims are intended to cover all such modifications and changes as falling within the true spirit of the invention.

(M6) The present disclosure may be implemented not only in a form of the pulse wave measurement method, but also in other various forms, such as programs enabling a computer to serve as the above-described pulse wave measurement method, a non-transitory, tangible computer-readable storage medium storing these programs, and a signal measurement apparatus.

What is claimed is:

1. A method for measuring a biometric signal of a subject, comprising:
repeatedly detecting the biometric signal using a biometric sensor;
detecting a noise intensity of noise contaminating the detected biometric signal;
adjusting a signal intensity of the detected biometric signal if the signal intensity meets an intensity change condition; and
withholding adjustment of the signal intensity if the detected noise intensity is greater than a noise threshold.

2. The method according to claim 1, wherein
the noise threshold comprises a body motion threshold,
the method further comprises repeatedly detecting an acceleration of a body of the subject using an acceleration sensor,
the detecting the noise intensity comprises detecting, as the noise intensity, a magnitude of a body motion of the subject from the detected acceleration, and
the withholding adjustment of the signal intensity comprises withholding adjustment of the signal intensity if the magnitude of the body motion is greater than the body motion threshold.

3. The method according to claim 2, wherein the detecting the noise intensity comprises detecting, as the noise intensity, a maximum magnitude of a body motion of the subject within a predetermined detection time.

4. The method according to claim 3, wherein the detection time is set to a period of time from a predetermined delay time before a current time to the current time.

5. The method according to claim 4, further comprising:
learning the delay time using the magnitude of the body motion and the biometric signal; and
setting the delay time to a learned value of the delay time.

6. The method according to claim 2, further comprising:
learning the body motion threshold using the magnitude of the body motion and the biometric signal; and
setting the body motion threshold to a learned value of the body motion threshold.

7. The method according to claim 1, wherein
the noise threshold comprises a signal threshold,
the method further comprises:
performing frequency analysis of the detected biometric signal; and
setting the signal threshold to a signal intensity in a frequency band intended for the biometric signal, based on a result of the frequency analysis of the detected biometric signal,
the detecting the noise intensity comprises detecting, as the noise intensity, a signal intensity outside of the frequency band intended for the biometric signal based on the result of the frequency analysis of the detected biometric signal, and
the withholding adjustment of the signal intensity comprises withholding adjustment of the signal intensity if the signal intensity outside of the frequency band intended for the biometric signal is greater than the signal threshold.

8. The method according to claim 1, wherein the biometric sensor is a pulse wave sensor configured to detect, as the biometric signal, a pulse wave signal.

9. The method according to claim 8, wherein
the pulse wave sensor comprises:
a light emitting element configured to irradiate an inside of a blood vessel of the subject with light;
a light receiving element configured to receive reflected light that is light emitted from the light emitting element and then reflected within the blood vessel and acquire the pulse wave signal from the reflected light,
the adjusting the signal intensity comprises adjusting an amount of light emitted from the light emitting element.

10. The method according to claim 1, wherein
the biometric sensor comprises an amplifier circuit configured to amplify a received biometric signal, and
the adjusting the signal intensity comprises adjusting a gain of the amplifier circuit.

11. The method according to claim 2, wherein
the biometric sensor is a pulse wave sensor configured to detect, as the biometric signal, a pulse wave signal, and
the acceleration sensor and the pulse wave sensor are two separate devices.

12. An apparatus for measuring a biometric signal of a subject, comprising:
a biometric sensor configured to repeatedly detect the biometric signal;
a controller configured to detect a noise intensity of noise contaminating the detected biometric signal, adjust a signal intensity of the detected biometric signal if the signal intensity meets an intensity change condition, and withhold adjustment of the signal intensity if the detected noise intensity is greater than a noise threshold.

13. The apparatus according to claim 12, wherein
the noise threshold comprises a body motion threshold,
the apparatus further comprises an acceleration sensor configured to repeatedly detect an acceleration of a body of the subject,
the controller is configured to detect, as the noise intensity, a magnitude of a body motion of the subject from the detected acceleration, and withhold adjustment of the signal intensity if the magnitude of the body motion is greater than the body motion threshold.

14. The apparatus according to claim 13, wherein the controller is configured to detect, as the noise intensity, a maximum magnitude of a body motion of the subject within a predetermined detection time.

15. The apparatus according to claim 14, wherein the detection time is set to a period of time from a predetermined delay time before a current time to the current time.

16. The apparatus according to claim 15, wherein the controller is further configured to learn the delay time using the magnitude of the body motion and the biometric signal, and set the delay time to a learned value of the delay time.

17. The apparatus according to claim 13, wherein the controller is further configured to learn the body motion threshold using the magnitude of the body motion and the biometric signal, and set the body motion threshold to a learned value of the body motion threshold.

18. The apparatus according to claim 12, wherein
the noise threshold comprises a signal threshold,
the controller is further configured to:
perform frequency analysis of the detected biometric signal,
set the signal threshold to a signal intensity in a frequency band intended for the biometric signal, based on a result of the frequency analysis of the detected biometric signal,
detect, as the noise intensity, a signal intensity outside of the frequency band intended for the biometric signal based on the result of the frequency analysis of the detected biometric signal, and
withhold adjustment of the signal intensity if the signal intensity outside of the frequency band intended for the biometric signal is greater than the signal threshold.

19. The apparatus according to claim 12, wherein the biometric sensor is a pulse wave sensor configured to detect, as the biometric signal, a pulse wave signal.

20. The apparatus according to claim 19, wherein
the pulse wave sensor comprises:
a light emitting element configured to irradiate an inside of a blood vessel of the subject with light;
a light receiving element configured to receive reflected light that is light emitted from the light emitting element and then reflected within the blood vessel and acquire the pulse wave signal from the reflected light,
the controller is further configured to adjust an amount of light emitted from the light emitting element, thereby adjusting the signal intensity.

21. The apparatus according to claim 12, wherein
the biometric sensor comprises an amplifier circuit configured to amplify a received biometric signal, and
the controller is further configured to adjust a gain of the amplifier circuit, thereby adjusting the signal intensity.

22. The apparatus according to claim 13, wherein
the biometric sensor is a pulse wave sensor configured to detect, as the biometric signal, a pulse wave signal, and
the acceleration sensor and the pulse wave sensor are two separate devices.
